(19) United States
(12) Patent Application Publication
(10) Pub. No.: US 2006/0078519 A1
(43) Pub. Date: Apr. 13, 2006

(54) Compositions comprising particles of at least one polymer dispersed in a fatty phase

(76) Inventors: Bertrand Lion, Paris (FR); Caroline Lebre, Thiais (FR); Veronique Ferrari, Maisons-Alfort (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,236

(22) Filed: Jun. 8, 2005

Related U.S. Application Data
(60) Provisional application No. 60/580,362, filed on Jun. 18, 2004.

(30) Foreign Application Priority Data
Jun. 8, 2005 (FR) 04 06173

(51) Int. Cl. A61K 8/00

(52) U.S. Cl. 424/70.1

(57) ABSTRACT

The present disclosure relates to cosmetic compositions comprising particles of at least one polymer dispersed in a fatty phase, wherein the fatty phase is free of volatile oil or comprises less than 50% by weight of volatile oil, relative to the weight of the fatty phase. The polymer may be such that when it is dispersed in the composition in sufficient amount, the composition is capable of forming a deposit having a resistance index of greater than or equal to 30%.

This application claims benefit of U.S. Provisional Application No. 60/580,362, filed Jun. 18, 2004, the contents of which are incorporated herein by reference. This application also claims benefit of priority under 35 U.S.C. § 119 to French Patent Application No. 04 06173, filed Jun. 8, 2004, the contents of which are also incorporated by reference.

The present disclosure relates to compositions, such as cosmetic compositions, comprising particles of at least one polymer dispersed in a fatty phase. These compositions may be used, for example, for making up and caring for the skin, including the scalp, of the human face and body, and human lips and integuments, for instance the hair, the eyelashes, and the eyebrows.

European Patent Application No. EP 1 002 528 discloses transfer-resistant compositions containing a dispersion of polymer particles in a volatile oil. These compositions predominantly contain a volatile oil, and also non-volatile silicone oils and non-volatile hydrocarbon-based oils. The non-volatile hydrocarbon-based oils are present in a very small amount, making up about 3% of the compositions.

There is still a need for cosmetic products that have good staying power and that are comfortable to wear, comprising particles of at least one polymer dispersed in a fatty phase. The inventors have found, surprisingly, that by using a dispersion of polymer particles in combination with a maximum amount of volatile oil, compositions that may have good staying power, while at the same time may be comfortable to wear, can be obtained.

In addition, the compositions with good staying power of the prior art often have the drawback of having little gloss. However, consumers nowadays are in search of products, for example lip and eyelid makeup products, that have good staying power and also a good level of gloss. In some embodiments, the compositions with good staying power according to the present disclosure may satisfy this need because they may also be glossy.

The compositions of the present disclosure may, for example, be comprised in hair products and makeup products for the human body, lips, and integuments that have care and/or treatment properties. For instance, these compositions may be comprised in lipsticks and lip glosses, eyeshadows, tattoo products, mascaras, eyeliners, artificial skin tanning products, optionally tinted care creams and protective creams, hair dye products, and haircare products.

In certain embodiments, the compositions with good staying power according to the present disclosure may be glossy, non-tacky, and may remain where initially deposited. When the compositions are applied to the skin or the lips, they may also afford a sensation of comfort on application and once deposited.

One embodiment of the present disclosure is, for example, a composition, such as a cosmetic composition, comprising particles of at least one polymer dispersed in a fatty phase, wherein the fatty phase is free of volatile oil or comprises less than 50% by weight of volatile oils relative to the weight of the fatty phase.

For example, the at least one polymer may be such that when it is dispersed in sufficient amounts in the compositions, the compositions may be capable of forming a deposit that has a resistance index of greater than or equal to 30%, for example greater than or equal to 40%, for instance greater than or equal to 45%, such as greater than or equal to 50%.

The resistance index of the deposit obtained with the compositions according to the present disclosure is determined according to the measuring protocol described below.

A support (40 mm×70 mm rectangle) comprising an acrylic coating (hypoallergenic acrylic adhesive on polyethylene film sold under the name Blenderme, ref. FH5000-55113, by the company 3M Santé) bonded onto a layer of polyethylene foam that is adhesive on the side opposite the one to which the adhesive plaster is fixed (foam layer sold under the name RE 40X70EP3 by the company Joint Technique Lyonnais Ind.) is prepared.

The color L*₀a*₀b*₀ of the support, on the acrylic coating side, is measured using a Minolta CR300 colorimeter.

The support thus prepared is preheated on a hotplate maintained at a temperature of 40° C. so that the surface of the support is maintained at a temperature of 330° C±1° C.

While leaving the support on the hotplate, the composition is applied to the entire non-adhesive surface of the support (i.e., to the surface of the acrylic coating), spreading it out with a brush to obtain a deposit of the composition of about 15 μm. This deposit is then left to dry for 10 minutes.

After drying, the color L*a*b* of the film thus obtained is measured.

The color difference ΔE1 between the color of the film relative to the color of the naked support is then determined via the following relationship:
ΔE1=\sqrt{(L*−L₀*)²+(a*−a₀*)²+(b*−b₀*)²}

The support is then bonded via its adhesive face (adhesive face of the foam layer) to an anvil 20 mm in diameter and equipped with a screw pitch. A sample of the support/deposit assembly is then cut out using a sample punch 18 mm in diameter. The anvil is then screwed onto a press (Statif Manuel Imada SV-2 from the company Someco) equipped with a tensile testing machine (Imada DPS-20 from the company Someco).

A strip 33 mm wide and 29.7 cm long is drawn on a sheet of white photocopier paper with a basis weight of 80 g/m², a first line is marked 2 cm from the edge of the sheet, and a second line is then marked 5 cm from the edge of the sheet, the first and second lines thus delimiting a box on the strip; next, a first mark and a second mark located in the strip at reference points 8 cm and 16 cm, respectively, from the second mark, are applied. 20 μl of water are placed on the first mark and 10 μl of refined sunflower oil (sold by the company Lesieur) are placed on the second mark.

The white paper is placed on the base of the press. The sample placed on the box of the strip of paper is then pressed at a pressure of about 300 g/cm² exerted for 30 seconds. The press is then opened and the sample is again placed just after the second mark (i.e., next to the box). A pressure of about 300 g/cm² is again exerted, and the paper is displaced, in a rectilinear manner as soon as the contact is made, at a speed of 1 cm/s over the entire length of the strip such that the sample passes through the water and oil deposits.

After removing the sample, some of the deposit has transferred onto the paper. The color L*′, a*′, b*′ of the deposit remaining on the sample is then measured.

The color difference ΔE2 between the color of the deposit remaining on the sample relative to the color of the naked support is then determined via the following relationship
ΔE2=\sqrt{(L*′−L₀*)²+(a*′−a₀*)²+(b*′−b₀*)²}

The resistance index of the composition, expressed as a percentage, is equal to the ratio:
100×ΔE2/ΔE1

The measurement is performed on 6 supports in succession, and the resistance index corresponds to the mean of the six measurements obtained with the six supports.

In certain embodiments, the polymer may be such that, when it is dispersed in a certain amount in, the composition, the mean gloss at 20° C. of a deposit of the composition, once spread onto a support, is greater than or equal to 30 out of 100.

The term “mean gloss,” as used herein, is understood to mean the gloss as conventionally measured using a glossmeter by the following method.

A coat ranging from 50 μm to 150 μm in thickness of the composition is spread using an automatic spreader onto a Leneta brand contrast card of reference Form 1 A Penopac. The coat covers at least the white background of the card. When the composition is solid, it is melted, if necessary, on the card after it has been spread so that it covers the white background.

The deposit is left to dry for 24 hours at a temperature of 30° C., and the gloss at 20° C. is then measured on the white background using a Byk Gardner brand glossmeter of reference microTri-Gloss.

This measurement (ranging from 0 to 100) is repeated at least three times, and the mean gloss is the mean of the at least three measurements taken.

The mean gloss of the compositions according to the present disclosure measured at 20° C. may be greater than or equal to 30, for example greater than or equal to 35, for instance greater than or equal to 40, such as greater than or equal to 45, greater than or equal to 50 out of 100, greater than or equal to 55, or greater than or equal to 60.

In certain embodiments, the mean gloss of the compositions, once spread onto a support, measured at 60° C. may be greater than or equal to 50, for example greater than or equal to 60, for instance greater than or equal to 65, such as greater than or equal to 70, greater than or equal to 75, greater than or equal to 80, greater than or equal to 85, or greater than or equal to 90 out of 100.

The mean gloss at 60° C. is measured as described above, by taking the measurement at 60° C. rather than at 20° C.

In one embodiment, the mean gloss of the compositions measured at 20° C. is greater than or equal to 35, for example 40, 45, or 50 out of 100, and/or the mean gloss of the compositions measured at 60° C. is greater than or equal to 65, for example 70 or 75 out of 100. In this embodiment, the compositions may constitute a liquid lipstick.

The present disclosure also relates to the use of at least one polymer dispersed in a fatty phase such that it is free of volatile oil or comprises less than 50% by weight of volatile oil relative to the weight of the fatty phase, to give gloss and/or staying power to a deposit of composition on the skin and/or the lips and/or the integuments, for example to a deposit of a makeup.

The term “fatty phase,” as used herein, is understood to mean any non-aqueous medium.

In certain embodiments, the fatty phase may be at least partially liquid at room temperature (25° C.) and atmospheric pressure (760 mmHg) and may be composed of at least one fatty substance that is liquid at room temperature, also known as an oil.

The present disclosure also relates to processes for making up the skin and/or the lips and/or the integuments, comprising applying the composition according to the present disclosure.

Polymer in Dispersion

According to the present disclosure, the at least one polymer may be a solid that is insoluble in the fatty phase of the compositions at room temperature, for example at approximately 25° C. The at least one polymer may also be insoluble in the fatty phase at its softening point, unlike a wax, which is soluble in the fatty phase at a temperature above its melting point. In this embodiment, the polymer is not a wax.

The at least one polymer in dispersion may also allow the formation of a deposit that may be continuous and homogeneous and/or is characterized by the over-lapping of the polymer chains.

The compositions according to the present disclosure may comprise at least one stable dispersion of spherical polymer particles of at least one polymer, in the fatty phase.

These dispersions may be in the form of polymer nanoparticles in stable dispersion in a liquid organic phase. The nanoparticles may have a mean size ranging from 5 to 800 nm, for example from 50 to 500 nm. However, it is possible to obtain polymer particles ranging up to 1 μm in size.

In certain embodiments, the polymer particles in dispersion may be insoluble in water-soluble alcohols, for instance ethanol.

The at least one polymer in dispersion that may be used in the compositions according to the present disclosure may have a molecular weight ranging from 2,000 to 10,000,000 g/mol and a Tg ranging from −100° C. to 300° C., for example from −50° C. to 100° C., such as from −10° C. to 50° C.

The at least one polymer may be film-forming. It is possible to use film-forming polymers having a low Tg, for example a Tg of less than or equal to skin temperature, such as less than or equal to 40° C.

The term “film-forming polymer,” as used herein, is understood to mean a polymer that is capable, by itself or in the presence of an auxiliary film-forming agent, of forming a continuous film that adheres to a support, for example to keratin materials, and may be a cohesive film, such as a film whose cohesion and mechanical properties are such that the film can be isolated from the support.

However, it may also be possible to use a non-film-forming polymer. The term “non-film-forming polymer,” as used herein, is understood to mean a polymer that is incapable by itself of forming an isolable film.

Among the film-forming polymers that may be used, non-limiting mention may be made of acrylic and vinyl free-radical homopolymers and copolymers, for example those with a Tg of less than or equal to 40° C., such as a Tg ranging from −10° C. to 30° C., used alone or as a mixture.

Among the non-film-forming polymers that may be used, non-limiting mention may be made of optionally crosslinked vinyl and acrylic free-radical homopolymers and copolymers, for example those with a Tg of greater than 40° C., such as a Tg ranging from 45° C. to 150° C., used alone or as a mixture.

The term “free-radical polymer,” as used herein, is understood to mean a polymer obtained by polymerization of unsaturated, for example ethylenic, monomers, each monomer being capable of homopolymerizing (unlike polycondensates). The free-radical polymers may be, for example, vinyl polymers and copolymers, such as acrylic polymers.

The acrylic polymers may result from the polymerization of ethylenically unsaturated monomers comprising at least one acid group and/or esters of these acid monomers and/or amides of these acids.

Meth(acrylic)/(meth)acrylate copolymers, for example acrylic/acrylate copolymers such that the mass ratio of the acrylic units and of the acrylate units ranges from 0.1% to 40%, for example from 2% to 30%, such as from 5% to 20%, may be used in compositions according to the present disclosure.

Monomers bearing an acid group that may be used include, but are not limited to, α,β-ethylenic unsaturated carboxylic acids such as acrylic acid, (meth)acrylic acid, crotonic acid, maleic acid, and itaconic acid. For example, (meth)acrylic acid and crotonic acid may be used.

The acid monomer esters may be chosen from (meth)acrylic acid esters (also known as (meth)acrylates), for instance alkyl(meth)acrylates, for example of a C₁-C₂₀, such as C₁-C₈ alkyl, aryl(meth)acrylates, for example of a C₆-C₁₀ aryl, and hydroxyalkyl(meth)acrylates, for example of a C₂-C₆ hydroxyalkyl. Non-limiting examples of alkyl(meth)acrylates that may be mentioned include methyl, ethyl, butyl, isobutyl, 2-ethylhexyl, and lauryl(meth)acrylate. Non-limiting examples of hydroxyalkyl (meth)acrylates that may be mentioned include hydroxyethyl(meth)acrylate and 2-hydroxypropyl(meth)acrylate. Non-limiting examples of aryl(meth)acrylates that may be mentioned include benzyl and phenyl acrylate.

Non-limiting examples of the (meth)acrylic acid esters that may be mentioned include the alkyl(meth)acrylates.

Among the free-radical polymers that may be used, non-limiting examples include copolymers of (meth)acrylic acid and of alkyl(meth)acrylate, such as of a C₁-C₄ alkyl. For example, ethyl acrylates optionally copolymerized with acrylic acid may be used.

Among the amides of the acid monomers that may be used, non-limiting mention may be made of (meth)acrylamides, for example N-alkyl(meth)acrylamides, for instance of a C₂-C₁₂ alkyl, such as N-ethylacrylamide, N-t-butylacrylamide, and N-octylacrylamide; and N-di(C₁-C₄)alkyl(meth)acrylamides.

The acrylic polymers may also result from the polymerization of ethylenically unsaturated monomers comprising at least one amine group, in free form or in partially or totally neutralized form, or alternatively in partially or totally quaternized form. Such monomers may be chosen from, for example, dimethylaminoethyl(meth)acrylate, dimethylaminoethyl(meth)acrylamide, vinylamine, vinylpyridine, and diallyldimethylammonium chloride.

The vinyl polymers may also result from the homopolymerization or copolymerization of at least one monomer chosen from vinyl esters and styrene monomers. For example, the at least one monomer may be polymerized with acid monomers and/or esters thereof and/or amides thereof, such as those mentioned above. Non-limiting examples of vinyl esters that may be mentioned include vinyl acetate, vinyl propionate, vinyl neodecanoate, vinyl pivalate, vinyl benzoate, and vinyl t-butylbenzoate. Non-limiting examples of styrene monomers that may be mentioned include styrene and a-methylstyrene.

The list of monomers given is not limiting, and it is possible to use any monomer known to those skilled in the art included in the categories of acrylic and vinyl monomers, including monomers modified with a silicone chain.

As other vinyl monomers that may be used, non-limiting mention may also be made of:
N-vinylpyrrolidone, N-vinylcaprolactam, vinyl-N-(C₁-C₈)alkylpyrroles, vinyloxazoles, vinylthiazoles, vinylpyrimidines, and vinylimidazoles,
olefins such as ethylene, propylene, butylene, isoprene, and butadiene.

The vinyl polymer may be crosslinked with at least one difunctional monomer, for example a difunctional monomer comprising at least two ethylenic unsaturations, such as ethylene glycol di(meth)acrylate and diallyl phthalate.

In a non-limiting manner, the polymers in dispersion comprised in compositions according to the present disclosure may be chosen from the following polymers and copolymers: polyurethanes, polyurethane-acrylics, polyureas, polyurea-polyurethanes, polyester-polyurethanes, polyether-polyurethanes, polyesters, polyesteramides, alkyds; acrylic polymers and copolymers; vinyl polymers and copolymers; acrylic-silicone copolymers; polyacrylamides; silicone polymers, for instance silicone poly-urethanes and silicone acrylics; fluoro polymers; and mixtures thereof.

The at least one polymer in dispersion in the fatty phase may represent from 5% to 40%, for example from 5% to 35%, such as from 8% to 30% by weight, relative to the weight of solids in the composition.

In one embodiment, the polymer particles in dispersion are surface-stabilized with a stabilizer that is solid at room temperature. In this case, the amount of solids in the dispersion may represent the total amount of polymer plus stabilizer, given that the amount of polymer cannot be less than 5%.

For example, a dispersion of particles of at least one film-forming polymer may be used in compositions according to the present disclosure.

Stabilizer

The polymer particles in organic medium may be surface-stabilized, as the polymerization proceeds, by means of at least one stabilizer that may be chosen from block polymers, grafted polymers, random polymers, and mixtures thereof. The stabilization may take place by any known means, for example by direct addition of the block polymer, grafted polymer, and/or random polymer during the polymerization.

The at least one stabilizer may also be present in the mixture before polymerization. However, it is also possible to add it continuously, for example when the monomers are also added continuously.

The at least one stabilizer may be present in an amount ranging from 2% to 30% by weight, for example from 5% to 20% by weight, relative to the weight of the initial monomer mixture.

When a grafted polymer and/or a block polymer is used as a stabilizer, the synthesis solvent may be chosen such that at least some of the grafts or blocks of the polymer-stabilizer are soluble in the solvent, the rest of the grafts or blocks being insoluble therein. The polymer-stabilizer used during the polymerization should be soluble, or dispersible, in the synthesis solvent. Furthermore, a stabilizer whose insoluble blocks or grafts have a certain affinity for the polymer formed during the polymerization may be chosen.

Among the grafted polymers, non-limiting mention may be made of silicone polymers grafted with a hydrocarbon-based chain and hydrocarbon-based polymers grafted with a silicone chain.

Grafted copolymers comprising, for example, an insoluble polyacrylic skeleton with poly(12-hydroxystearic acid) soluble grafts are also suitable for use.

Thus, grafted-block or block copolymers comprising at least one polyorganosiloxane block and at least one block of a free-radical polymer, for instance grafted acrylic/silicone copolymers, may be used, for example when the synthesis medium and then the organic phase of the first composition contains a silicone phase.

It is also possible to use grafted-block and block copolymers comprising at least one polyorganosiloxane block and at least one block of a polyether. The polyorganosiloxane block may be chosen from polydimethylsiloxanes and poly(C₂-C₁₈)alkylmethylsiloxanes; the polyether block may be chosen from poly(C₂-C₁₈)alkylenes, such as polyoxyethylene and/or polyoxypropylene. For example, dimethicone copolyols and (C₂-C₁₈)alkyldimethicone copolyols such as those sold under the name “Dow Corning 3225C” by the company Dow Corning, and lauryl methicones such as those sold under the name “Dow Corning Q2-5200” by the company Dow Corning, may be used.

Further non-limiting examples of grafted-block and block copolymers that may be used include those comprising at least one block resulting from the polymerization of at least one ethylenic monomer comprising at least one optionally conjugated ethylenic bond, for instance ethylene and dienes such as butadiene and isoprene, and of at least one block of a vinyl polymer or a styrene polymer. When the ethylenic monomer comprises several optionally conjugated ethylenic bonds, the residual ethylenic unsaturations after the polymerization may be hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these polymers that may be used, non-limiting mention may be made of block copolymers, for example of diblock copolymers and triblock copolymers, for instance polystyrene/polyisoprene (SI), polystyrene/polybutadiene (SB), such as those sold under the name “Luvitol HSB” by BASF, of the type such as polystyrene/copoly(ethylene-propylene) (SEP), for example those sold under the name “Kraton” by Shell Chemical Co., and of the type such as polystyrene/copoly(ethylene-butylene) (SEB). For example, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), and Kraton D-1107 (SIS) may be used. The polymers may be known as hydrogenated or non-hydrogenated diene copolymers.

Gelled Permethyl 99A-750, 99A-753-59, and 99A-753-58 (mixture of triblock and of star polymer), Versagel 5960 from Penreco (triblock plus star polymer); and OS129880, OS129881, and OS84383 from Lubrizol (styrene/(meth)acrylate copolymer) may also be used.

Among grafted-block and block copolymers comprising at least one block resulting from the polymerization of at least one ethylenic monomer comprising at least one ethylenic bond and of at least one block of an acrylic polymer, non-limiting mention may be made of poly(methyl(meth)acrylate)/polyisobutylene diblock and triblock copolymers and grafted copolymers comprising a poly(methyl(meth)acrylate) skeleton and polyisobutylene grafts.

Among grafted-block and block copolymers comprising at least one block resulting from the polymerization of at least one ethylenic monomer comprising at least one ethylenic bond and of at least one block of a polyether such as a C₂-C₁₈ polyalkylene, for example polyethylene and/or polyoxypropylene, non-limiting mention may be made of polyoxyethylene/polybutadiene and polyoxyethylene/polyisobutylene diblock and triblock copolymers.

When a random polymer is used as stabilizer, it may be chosen such that it has a sufficient amount of groups to make it soluble in the intended organic synthesis medium.

Copolymers based on alkyl acrylates and (meth)acrylates derived from C₁-C₄ alcohols and on alkyl acrylates and (meth)acrylates derived from C₈-C₃₀ alcohols may thus be used. Non-limiting mention may be made of stearyl(meth)acrylate/methyl(meth)acrylate copolymer.

When the synthesis medium is apolar, one may choose as a stabilizer a polymer that provides the fullest possible coverage of the particles, several polymer-stabilizer chains then being adsorbed onto a particle of polymer obtained by polymerization.

In this embodiment, one may use as a stabilizer either a grafted polymer or a block polymer, so as to have better interfacial activity. For example, blocks or grafts that are insoluble in the synthesis solvent provide bulkier coverage at the surface of the particles.

When the liquid synthesis solvent comprises at least one silicone oil, the stabilizer may be chosen from grafted-block and block copolymers comprising at least one polyorganosiloxane block and at least one block of a free-radical polymer or of a polyether or of a polyester, for instance polyoxypropylene and/or polyoxyethylene blocks.

When the liquid organic phase does not comprise any silicone oil, the at least one stabilizer may be chosen from:
(a) grafted-block and block copolymers comprising at least one polyorganosiloxane block and at least one block of a free-radical polymer or of a polyether or a polyester,
(b) copolymers of alkyl acrylates or (meth)acrylates derived from C₁-C₄ alcohols and of alkyl acrylates or (meth)acrylates derived from C₈-C₃₀ alcohols,
(c) grafted-block and block copolymers comprising at least one block resulting from the polymerization of at least one ethylenic monomer comprising conjugated ethylenic bonds, and at least one block of a vinyl or acrylic polymer or of a polyether or of a polyester, or mixtures thereof.

For example, diblock polymers may be used as a stabilizer.

Plasticizer

In one embodiment, when the polymer has a glass transition temperature that is too high for the intended application, a plasticizer may be combined therewith. The plasticizer may be chosen from the plasticizers usually used in the art, for example from compounds liable to be solvents for the polymer. Coalescers may also be used in order to aid the polymer to form a continuous and homogeneous deposit.

Non-limiting examples of coalescers or plasticizers that may be used according to the present disclosure include those mentioned in French Patent Application No. FR 2782917.

The compositions according to the present disclosure may comprise at least one ester of at least one carboxylic acid comprising from 1 to 7 carbon atoms and of a polyol comprising at least four hydroxyl groups, the ester having a molar mass of less than 5,000 g/mol.

The polyol may be chosen from monosaccharides and polysaccharides comprising from 1 to 10 saccharides, for example from 1 to 4, such as one or two saccharides.

For example, the polyol according to the present disclosure may be a disaccharide. Among the disaccharides that may be used in compositions according to the present disclosure, non-limiting mention may be made of sucrose (α-D-glucopyranosyl-(1-2)-β-D-fructofuranose), lactose (β-D-galactopyranosyl-(1-4)-β-D-glucopyranose), and maltose (α-D-glucopyranosyl-(1-4)-β-D-glucopyranose).

In one embodiment, the ester is sucrose diacetate hexakis(2-methylpropanoate).

The ester may be liquid at room temperature and atmospheric pressure. It may be present in an amount ranging from 0.1% to 25% by weight, for example from 0.5% to 15% by weight, such as from 3% to 15% by weight, relative to the total weight of the composition.

The mass ratio between the polymer particles and the ester of acid and polyol may range from 0.5 to 100, for example from 1 to 50, such as from 1 to 10 and from 1 to 5.

Fatty Phase

The fatty phase of the compositions according to the present disclosure may comprise at least one cosmetically and/or dermatologically acceptable and generally physiologically acceptable oil, chosen, for example, from carbon-based oils, hydrocarbon-based oils, fluoro oils and/or silicone oils of mineral, plant, and synthetic origin, and mixtures thereof.

As used herein, the term “oil” is understood to mean any non-aqueous medium that is liquid at room temperature (25° C.) and atmospheric pressure (760 mmHg).

The total fatty phase may be present in compositions according to the present disclosure in an amount ranging from 5% to 90%, for example from 20% to 85% by weight, relative to the total weight of the composition. For example, it may be present in an amount of at least 30% by weight, relative to the total weight of the composition.

In one embodiment, the fatty phase is free of or comprises less than 50% by weight of at least one volatile oil relative to the total weight of the fatty phase. For example, the fatty phase may comprise less than 40%, for instance less than 30%, such as less than 20% or such as less than 10% by weight of at least one volatile oil, relative to the total weight of the fatty phase.

Volatile Oil of the Fatty Phase

At least one volatile oil may be included in the fatty phase of the compositions according to the present disclosure, provided that it represents less than 50% by weight of the fatty phase. The at least one oil may be chosen from hydrocarbon-based oils and silicone oils optionally comprising alkyl or alkoxy groups that are pendent or at the end of a silicone chain.

The term “volatile oil,” as used herein, is understood to mean any oil having a vapor pressure, at room temperature and atmospheric pressure, of greater than 0.02 mmHg.

Among volatile silicone oils that may be used in compositions according to the present disclosure, non-limiting mention may be made of linear and cyclic silicones having a viscosity at room temperature of less than 8 cSt, for example comprising from 2 to 7 silicon atoms, these silicones optionally comprising alkyl or alkoxy groups comprising from 1 to 10 carbon atoms. As volatile silicone oils that may be used according to the present disclosure, non-limiting mention may be made of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, and mixtures thereof.

Among volatile oils that may be used in the present disclosure, non-limiting mention may be made of C₈-C₁₆ isoalkane oils (also known as isoparaffins), for instance isododecane, isodecane, and isohexadecane and, for example, the oils sold under the trade names Isopar and Permethyl, such as isododecane (Permethyl 99A).

In certain embodiments, the fatty phase of the compositions may comprise less than 10% of at least one volatile oil. For example, the fatty phase may comprise less than 5%, less than 3%, or even less than 1% of at least one volatile oils.

Apolar or Sparingly Polar Oil

In certain embodiments, the fatty phase may comprise at least one apolar or sparingly polar oil, which can be present in an amount of at least 5% by weight, relative to the total weight of the composition. For example, the at least one apolar or sparingly polar oil of the fatty phase may be a non-volatile hydrocarbon-based apolar or sparingly polar oil, which may be hydrocarbon-based.

In certain embodiments, the apolar oils may have a solubility parameter δₐ=0.

The term “polar oil,” as used herein, is understood to mean an oil composed of chemical compounds comprising at least one polar group. The “polar groups” may be those well known to those skilled in the art; they may be, for example, chosen from ionic polar groups and nonionic groups chosen from —COOH; —OH; ethylene oxide; propylene oxide; —PO₄; —NHR; and —NR₁R₂, with R₁ and R₂ optionally forming a ring and representing a linear or branched C₁ to C₂₀ alkyl or alkoxy radical.

The at least one sparingly polar oil comprises oils that have a mean solubility parameter at 25° C. of: 0<δₐ<5.0 (J/cm³)^{1/2}.

The at least one highly polar oil has a mean solubility parameter δₐ according to the Hansen solubility space, at 25° C., of: δₐ≧5.0 (J/Cm³)^{1/2}.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)^{1/2}.

The parameters δₚ, δₕ, δ_{D} and δₐ are expressed in (J/cm³)^{1/2}.

When the oily phase is a mixture of different oils, the solubility parameters of the mixture may be determined from those of the compounds taken separately, according to the following relationships:
δ_{Dmixt}=Σxi δ_{Di}; δₚₘᵢₓₜ=Σxi δₚᵢ and δₕₘᵢₓₜ=ρxi δₕᵢ
δₐₘᵢₓₜ=(δ²ₚₘᵢₓₜ+δ²ₕₘᵢₓₜ)^{1/2}
wherein xi represents the volume fraction of compound i in the mixture.

A person skilled in the art may determine the amounts of each oil to obtain an oily phase that satisfies the desired criteria.

The at least one apolar or sparingly polar oil may be hydrocarbon-based. The term “hydrocarbon-based oil,” as used herein, is understood to mean an oil formed from, or comprising, carbon and hydrogen atoms, and optionally oxygen and nitrogen atoms, and comprising no silicon or fluorine atoms. It may comprise at least one group chosen from alcohol, ester, ether, carboxylic acid, amine, and amide groups.

The at least one apolar or sparingly polar oil may be non-volatile. The term “non-volatile oil,” as used herein, is understood to mean any oil having a non-zero vapor pressure at room temperature and atmospheric pressure, of less than 0.02 mmHg, such as less than 10⁻³ mmHg.

The at least one apolar or sparingly polar oil may be present in an amount of at least 5% by weight, relative to the total weight of the composition. For example, the apolar or sparingly polar oil may be present in an amount ranging from 5% to 80% by weight, relative to the total weight of the composition, for instance from 10% to 60% by weight, such as from 10% to 40% by weight, relative to the total weight of the composition.

The at least one hydrocarbon-based apolar or sparingly polar oil may represent from 10% to 40% by weight, for example from 15% to 30% by weight, relative to the total weight of the composition.

In certain embodiments, the at least one apolar or sparingly polar oil may be a non-volatile apolar hydrocarbon-based oil.

In one embodiment, the at least one apolar hydrocarbon-based oil comprises no heteroatoms. The term “heteroatom,” as used herein, is understood to mean an atom other than carbon or hydrogen.

In one embodiment, the at least one non-volatile hydrocarbon-based apolar oil is chosen from linear and branched saturated alkanes.

The at least one non-volatile apolar or sparingly polar hydrocarbon-based oil may be chosen from hydrocarbon-based oils with a molar mass ranging from 300 to 900 g/mol, for example from 350 to 800 g/mol.

In one embodiment, the at least one non-volatile hydrocarbon-based apolar oil is chosen from linear and branched hydrocarbons, such as liquid paraffin, liquid petroleum jelly, and liquid naphthalene, hydrogenated polyisobutene, isoeicosane, squalane, and decene/butene copolymers, and mixtures thereof.

In one embodiment, the fatty phase comprises from 30% to 70% by weight of at least one apolar non-volatile hydrocarbon-based oil, relative to the weight of the fatty phase, for example from 40% to 60% by weight, relative to the weight of the fatty phase.

As further examples of non-volatile apolar or sparingly polar oils, non-limiting mention may be made of:
hydrocarbon-based oils of animal origin, for instance squalene;
hydrocarbon-based plant oils such as liquid triglycerides of fatty acids comprising at least 10 carbon atoms;
synthetic esters and ethers, for example of fatty acids, for instance the oils of formula R₁(CO)ₓOR₂ wherein R₁ is chosen from acid residues comprising from 2 to 29 carbon atoms with x being 0 or 1 and R₂ is chosen from hydrocarbon-based chains comprising from 3 to 30 carbon atoms, for instance tributyl acetyl citrate, oleyl erucate, 2-octyidodecyl behenate, triisoarachidyl citrate, isocetyl stearoylstearate, octyldodecanyl stearoylstearate, n-propyl acetate, tridecyl trimellitate, diisocetyl dodecanedioleate, diisocetyl stearate, arachidyl propionate, dibutyl phthalate, propylene carbonate, and octyldodecyl pentanoate; and polyol esters, for instance vitamin F, sorbitan isostearate, glyceryl triisostearate, and diglyceryl triisostearate; and
mixtures thereof.

The fatty phase of the compositions may also comprise at least one non-volatile silicone oil chosen from:
polydimethylsiloxanes (PDMS) optionally comprising an entity chosen from C₃-C₄₀ alkyl groups, C₃-C₄₀ alkoxy chains, and phenyl radicals; the polydimethylsiloxanes comprising phenyl radicals may be chosen from phenyl trimethicones;
optionally fluorinated polyalkylmethylsiloxanes, for instance polymethyltrifluoropropyldimethylsiloxanes,
polyalkylmethylsiloxanes substituted with functional groups such as hydroxyl, thiol, and/or amine groups; and
polysiloxanes modified with fatty acids, fatty alcohols, or polyoxyalkylenes.

In certain embodiments, the fatty phase of the compositions may comprise less than 10% of at least one silicone oils. For example, the fatty phase may comprise less than 5%, less than 3%, or even less than 1% of at least one silicone oils.

Non-Volatile Highly Polar Oil

The fatty phase may comprise, in addition to the at least one apolar or sparingly polar oil as described above, at least one highly polar non-volatile oil chosen from fatty acid esters comprising from 7 to 29 carbon atoms, for instance diisostearyl malate, isopropyl palmitate, diisopropyl adipate, caprylic/capric acid triglycerides, for instance those sold by the company Stearineries Dubois and those sold under the names Miglyol 810, 812, and 818 by the company Dynamit Nobel, Shea butter oil, isopropyl myristate, butyl stearate, hexyl laurate, diisopropyl adipate, isononyl isononate, 2-hexyldecyl laurate, 2-octyldecyl palmitate, 2-octyldodecyl myristate, 2-octyldodecyl lactate, 2-diethylhexyl succinate, 2-ethylhexyl palmitate, 2-octyldodecyl stearate, and castor oil; lanolic acid, lauric acid, and stearic acid esters; higher fatty alcohols comprising from 7 to 29 carbon atoms, such as stearyl alcohol, linoleyl alcohol, linolenyl alcohol, isostearyl alcohol, 2-octyldodecanol, decanol, dodecanol, octadecanol, and oleyl alcohol; higher fatty acids comprising from 7 to 29 carbon atoms, such as myristic acid, palmitic acid, stearic acid, behenic acid, oleic acid, linoleic acid, linolenic acid, and isostearic acid; and mixtures thereof.

This at least one non-volatile highly polar oil may be present in an amount ranging from 0.1% to 10%, for example from 1% to 5% by weight, relative to the total weight of the composition.

Oil of High Molar Mass

In one embodiment, the fatty phase may comprise, in addition to the at least one apolar or sparingly polar oil, at least one oil of high molar mass, for example ranging from 650 to 10,000 g/mol.

The compositions according to the present disclosure may comprise an amount ranging from 2% to 30%, for example from 5% to 25%, such as from 5% to 15% of at least one oil with a molar mass ranging from 650 to 10,000 g/mol, for instance ranging from 750 to 7,500 g/mol.

In one embodiment, the at least one oil with a molar mass ranging from 650 to 10,000 g/mol may be chosen from:
polybutylenes such as Indopol H-100 (molar mass or MM=965 g/mol), Indopol H-300 (MM=1,340 g/mol) and Indopol H-1500 (MM=2,160 g/mol), sold or manufactured by the company Amoco,
hydrogenated polyisobutylenes such as Panalane H-300 E sold or manufactured by the company Amoco (M=1,340 g/mol), Viseal 20 000 sold or manufactured by the company Synteal (MM=6,000 g/mol) and Rewopal PIB 1000 sold or manufactured by the company Witco (MM=1,000 g/mol),
polydecenes and hydrogenated polydecenes such as Puresyn 10 (MM=723 g/mol) and Puresyn 150 (MM=9,200 g/mol), sold or manufactured by the company Mobil Chemicals,
vinylpyrrolidone copolymers such as the vinylpyrrolidone/1-hexadecene copolymer Antaron V-216 sold or manufactured by the company ISP (MM=7,300 g/mol), esters such as:
   a) linear fatty acid esters with a total carbon number ranging from 35 to 70, for instance pentaerythrityl tetrapelargonate (MM=697.05 g/mol);
   b) hydroxylated esters such as polyglyceryl-2 triisostearate (MM=965.58 g/mol),
   c) aromatic esters such as tridecyl trimellitate (MM=757.19 g/mol);
   d) esters of branched C₂₄-C₂₈ fatty alcohol or fatty acids, such as those described in European Patent Application No. EP 0 955 039, for example triisoarachidyl citrate (MM=1,033.76 g/mol), pentaerythrityl tetraisononanoate (MM=697.05 g/mol), glyceryl triisostearate (MM=891.51 g/mol), glyceryl tris(2-decyl)tetradecanoate (MM=1,143.98 g/mol), pentaerythrityl tetraisostearate (MM=1,202.02 g/mol), polyglyceryl-2 tetraisostearate (MM=1,232.04 g/mol), and pentaerythrityl tetrakis(2-decyl)tetradecanoate (MM=1,538.66 g/mol); and
   e) diol dimer esters and polyesters, such as esters of a diol dimer and of a fatty acid, and esters of a diol dimer and of a diacid.
   The esters of a diol dimer and of a monocarboxylic acid may be obtained from a monocarboxylic acid comprising from 4 to 34 carbon atoms, for example from 10 to 32 carbon atoms, these acids being linear or branched, and saturated or unsaturated.
   As illustrative examples of monocarboxylic acids that are suitable for use in the compositions according to the present disclosure, non-limiting mention may be made of fatty acids.
   The esters of a diol dimer and of a dicarboxylic acid may be obtained from a diacid dimer derived, for example, from the dimerization of an unsaturated fatty acid, for instance of C₈ to C₃₄, of C₁₂ to C₂₂, of C₁₈ to C₂₀, or of C₁₈.
   In one embodiment, the diacid dimer is that from which the diol dimer to be esterified is also derived.
   The diol dimer esters may be obtained from a diol dimer produced by catalytic hydrogenation of a diacid dimer as described above, for example hydrogenated dilinoleic diacid.
   As illustrations of diol dimer esters, non-limiting mention may be made of the esters of dilinoleic diacids and of dilinoleyl diol dimers sold by the company Nippon Fine Chemical under the trade name Lusplan DD-DA5® and DD-DA7®.
silicone oils such as phenylsilicones, for instance Belsil PDM 1000 from the company Wacker (MM=9,000 g/mol), and
oils of plant origin such as sesame oil (820.6 g/mol),
and mixtures thereof.
   Synthesis Medium for the Polymer Particles

In one embodiment, the fatty phase of the compositions comprises at least one oil, which is the at least one organic solvent serving as the polymerization medium for the polymer particles as described above.

The polymer dispersion may be manufactured as described in European Patent Application No. EP 0 749 747.

In this embodiment, a mixture comprising the initial monomers and a free-radical initiator is also prepared. This mixture is dissolved in a solvent, which is referred to in the description hereinbelow as the “synthesis solvent”.

A synthesis solvent may be chosen such that the initial monomers and the free-radical initiator may be soluble therein, and the polymer particles obtained may be insoluble therein, such that they precipitate therefrom during their formation. For example, the synthesis solvent may be chosen from alkanes such as heptane, isododecane, and cyclohexane. The polymerization of the polymer particles may be performed in a synthesis solvent as described above, and the at least one apolar or sparingly polar oil described above may then be added and the synthesis solvent may be selectively distilled off, provided that the at least one apolar or sparingly polar oil is miscible with the synthesis solvent.

The monomers may be present in the synthesis solvent, before polymerization, in an amount ranging from 5% to 20% by weight, relative to the weight of the reaction mixture. The total amount of the monomers may be present in the solvent before the start of the reaction, or some of the monomers may be added gradually as the polymerization reaction proceeds.

The free-radical initiator may be chosen from azobisisobutyronitrile and tert-butylperoxy-2-ethyl hexanoate.

Wax, Pasty Compound

The compositions according to the present disclosure may also comprise at least one wax.

As used herein, the term “wax” is understood to mean a lipophilic fatty compound that is solid at room temperature (25° C.), which undergoes a reversible solid/liquid change of state, having a melting point of greater than or equal to 30° C., and having an anisotropic crystal organization in the solid state. The size of the crystals may be such that the crystals diffract and/or scatter light, giving the compositions a cloudy, more or less opaque appearance. By bringing the wax to its melting point, it may be possible to make it miscible with oils and to form a microscopically homogeneous mixture, but, on returning the temperature of the mixture to room temperature, recrystallization of the wax in the oils of the mixture may be obtained.

For the purposes of the present disclosure, the melting point of the wax corresponds to the temperature of the most endothermic peak observed by thermal analysis (DSC) as described in standard ISO 11357-3; 1999.

The melting point of the wax may be measured using a differential scanning calorimeter (DSC), for example the calorimeter sold under the name MDSC 2920 by the company TA Instruments.

The measuring protocol is, for example, as follows:

A sample of 5 mg of wax placed in a crucible is subjected to a first temperature increase ranging from −20° C. to 100° C., at a heating rate of 10° C./minute, is then cooled from 100° C. to −20° C. at a cooling rate of 10° C./minute, and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the value of the temperature corresponding to the top of the peak of the curve representing the variation of the difference in power absorbed as a function of the temperature.

The term “apolar wax,” as used herein, is understood to mean a hydrocarbon-based or silicone apolar wax.

The at least one wax may be chosen from hydrocarbon-based waxes, fluoro waxes, and silicone waxes, and may be of plant, mineral, animal, and/or synthetic origin. For example, the at least one wax may have a melting point of greater than 45° C.

As examples of waxes that may be used in the compositions according to the present disclosure, non-limiting mention may be made of beeswax, carnauba wax, candelilla wax, paraffin, microcrystalline waxes, ceresin, and ozokerite; synthetic waxes, for instance polyethylene waxes and Fischer-Tropsch waxes; and silicone waxes, for instance alkyl and alkoxy dimethicones comprising from 16 to 45 carbon atoms.

The compositions according to the present disclosure may comprise at least one apolar wax such as a hydrocarbon-based or silicone apolar wax.

The compositions may comprise at least one apolar hydrocarbon-based wax. The term “apolar hydrocarbon-based wax,” as used herein, is understood to mean a wax comprising at least 95% by weight of chemical compounds free of polar groups. The “polar groups” may be well known to those skilled in the art; they may be, for example, ionic or nonionic polar groups chosen from —COOH; —-OH; ethylene oxide; propylene oxide; —PO₄; —NHR; and —NR₁R₂, with R₁ and R₂ optionally forming a ring and representing a linear or branched C₁ to C₂₀ alkyl or alkoxy radical.

In one embodiment, the hydrocarbon-based wax comprises at least 95% by weight of compounds free of heteroatoms.

In another embodiment, the apolar hydrocarbon-based wax comprises at least 95% by weight of chemical compounds comprising carbon and hydrogen. These chemical compounds may be chosen from linear and branched saturated alkanes.

In yet another embodiment, the apolar wax is chosen from linear hydrocarbon-based waxes.

Non-limiting examples of linear hydrocarbon-based waxes include ethylene polymers and copolymers, linear paraffin waxes, and Fischer-Tropsch waxes.

Non-limiting illustrations of hydrocarbon-based waxes that may be mentioned include Fischer-Tropsch waxes, which are also known as polymethylene waxes. They correspond to the formula CₙH₂ₙ₊₂.

In one embodiment, the at least one wax may be a polymethylene wax, for example the wax Cirebelle 505® manufactured by the company Sasol, with a melting point equal to 40° C.

The apolar wax may be a polyoxyalkylenated silicone wax, i.e., a silicone comprising at least one oxyalkylene group of the type (—CₓH₂ₓO)ₐ wherein x may range from 2 to 6 and a is greater than or equal to 2.

In one embodiment, the oxyalkylenated silicones that may be suitable for use in the compositions according to the present disclosure may be chosen from the compounds of general formulae (I), (II), (III), and (IV): 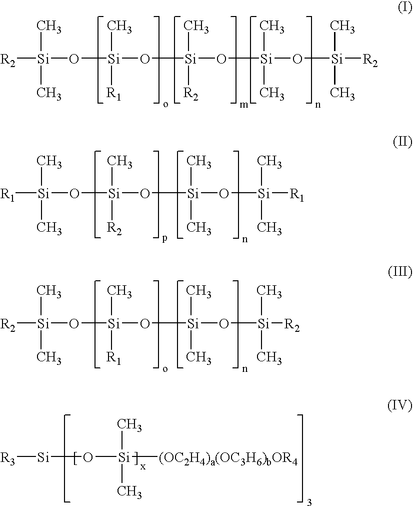
wherein:

R₁, which may be identical or different, is chosen from linear and branched C₁-C₃₀ alkyl radicals and phenyl radicals,

R₂, which may be identical or different, is chosen from C_{c}H_{2c}—O—(C₂H₄O)ₐ(C₃H₆O)_{b}R₅ radicals and —C_{c}H_{2c}—O—(C₄H₈O)ₐ—R₅ radicals,

R₃ and R₄, which may be identical or different, are chosen from linear and branched C₁ to C₁₂ alkyl radicals, for example methyl radicals,

R₅, which may be identical or different, is chosen from hydrogen, linear and branched alkyl radicals comprising from 1 to 12 carbon atoms, linear and branched alkoxy radicals comprising from 1 to 6 carbon atoms, linear and branched acyl radicals comprising from 2 to 30 carbon atoms, hydroxyl radicals, C₁-C₆ aminoalkoxy radicals optionally substituted on the amine, C₂-C₆ aminoacyl radicals optionally substituted on the amine, aminoalkyl radicals optionally substituted on the amine and on the alkyl chain, C₂-C₃₀ carboxyacyl radicals, groups optionally substituted with one or two substituted aminoalkyl radicals, —NHCO(CH₂)_{d}OH radicals, and phosphate groups,

d ranges from 1 to 10,

m ranges from 0 to 20,

n ranges from 0 to 500,

o ranges from 0 to 20,

p ranges from 1 to 50,

a ranges from 0 to 50,

b ranges from 0 to 50,

with the proviso that a +b is greater than or equal to 2,

c ranges from 0 to 4,

x ranges from 1 to 100.

Such silicones are described, for example, in U.S. Pat. Nos. 5,070,171, 5,149,765, 5,093,452, and 5,091,493.

Non-limiting examples of silicones that may be mentioned are those of formula (III), wherein R₂, which may be identical or different, is chosen from C_{c}H_{2c}—O—(C₂H₄O)ₐ(C₃H₆O)_{b}—R₅ radicals, with R₅, a, b, and c being defined as above. In this embodiment, b and c may be equal to 0 and a may range from 1 to 50, for example from 5 to 30, such as from 10 to 20.

The low-melting apolar wax may be present in an amount ranging from 1% to 30%, for example from 3% to 20% by weight, relative to the total weight of the composition.

The mass ratio between the polymer particles and the low-melting apolar wax may be range from 0.5 to 100, for example from 1 to 50, for instance from 1 to 20, such as from 3 to 15.

The compositions according to the present disclosure may also comprise at least one additional wax other than the low-melting apolar wax described above.

The at least one additional wax may have a melting point of greater than or equal to 65° C. It may be chosen from beeswax, carnauba wax, candelilla wax, paraffin, microcrystalline waxes, ceresin, ozokerite, polyethylene waxes, Fischer-Tropsch waxes, and mixtures thereof.

In one embodiment, the apolar wax with a melting point of less than 65° C. (a) and the wax whose melting point is greater than or equal to 65° C. (b) are in a mass proportion (a)/(b) ranging from 30/70 to 55/45, for example from 40/60 to 45/55.

The amount of all the waxes comprised in the compositions range from 15% to 35%, for example from 20% to 30% by weight, relative to the total weight of the composition.

The at least one additional wax may be chosen from apolar waxes with a melting point of greater than 65° C., for instance microcrystalline waxes, polyethylene waxes, paraffin waxes, and mixtures thereof.

The compositions according to the present disclosure may also comprise at least one pasty compound.

Hydrophilic Medium

The compositions according to the present disclosure may comprise a hydrophilic medium comprising water or a mixture of water and of at least one hydrophilic organic solvent, for instance alcohols, such as linear and branched lower monoalcohols comprising from 2 to 5 carbon atoms, for example ethanol, isopropanol, and n-propanol, and polyols, for instance glycerol, diglycerol, propylene glycol, sorbitol, pentylene glycol, and polyethylene glycols, and hydrophilic C₂ ethers and C₂-C₄ aldehydes.

The water or the mixture of water and of at least one hydrophilic organic solvent may be present in the compositions according to the present disclosure in an amount ranging from 0.1% to 99% by weight, for example from 10% to 80% by weight, relative to the total weight of the composition.

Semi-Crystalline Polymer

The compositions according to the present disclosure may comprise at least one semi-crystalline polymer.

As used herein, the term “polymers” is understood to mean compounds comprising at least two repeating units, for example at least three repeating units, such as at least ten repeating units.

As used herein, the term “semi-crystalline polymer” is understood to mean polymers comprising a crystallizable portion and an amorphous portion in the skeleton and having a first-order reversible change of phase temperature, such as of melting (solid-liquid transition). The crystallizable portion may be either a side chain (or pendent chain) or a block in the skeleton.

When the crystallizable portion of the at least one semi-crystalline polymer is a block of the polymer skeleton, this crystallizable block has a different chemical nature from that of the amorphous blocks; in this case, the semi-crystalline polymer may be a block copolymer, for example a diblock, triblock or multiblock copolymer. When the crystallizable portion is a chain that is pendent on the skeleton, the semi-crystalline polymer may be a homopolymer or a copolymer.

The terms “organic compound” and “having an organic structure,” as used herein, are understood to mean compounds comprising carbon atoms, hydrogen atoms, and optionally heteroatoms such as S, O, N, and P, alone or in combination.

The melting point of the at least one semi-crystalline polymer may be less than 150° C.

The melting point of the at least one semi-crystalline polymer may be greater than or equal to 30° C. and less than 100° C. For example, the melting point of the at least one semi-crystalline polymer may be greater than or equal to 30° C. and less than 60° C.

The at least one semi-crystalline polymer may be a solid at room temperature (25° C.) and atmospheric pressure (760 mmHg), with a melting point of greater than or equal to 30° C. The melting point values may correspond to the melting point measured using a differential scanning calorimeter (DSC), such as the calorimeter sold under the name DSC 30 by the company Mettler, with a heating rate of 5 or 10° C. per minute. In these measurements, the melting point corresponds to the temperature of the most endothermic peak of the thermogram.

The at least one semi-crystalline polymer may have a melting point that is higher than the temperature of the keratinous support, for example the skin and the lips, that the compositions according to the present disclosure may be used on.

The at least one semi-crystalline polymer may be capable, alone or as a mixture, of structuring the compositions without the addition of a particular surfactant or a filler or a wax.

According to the present disclosure, the at least one semi-crystalline polymer may be soluble in the fatty phase, for example to at least 1% by weight, at a temperature that is higher than its melting point. Apart from the crystallizable chains or blocks, the blocks of the polymers may be amorphous.

As used herein, the expression “crystallizable chain or block” is understood to mean a chain or block that, if it were obtained alone, would change from the amorphous state to the crystalline state reversibly, depending on whether one is above or below the melting point. As used herein, the term “chain” is understood to mean a group of atoms that are pendent or lateral relative to the polymer skeleton. The term “block,” as used herein, is understood to mean a group of atoms belonging to the skeleton, this group constituting one of the repeating units of the polymer.

The polymer skeleton of the semi-crystalline polymers may be soluble in the fatty phase.

In one embodiment, the crystallizable blocks or chains of the at least one semi-crystalline polymer represent at least 30% of the total weight of each polymer, for example at least 40%. The at least one semi-crystalline polymer with crystallizable side chains may be homopolymers or copolymers. The at least one semi-crystalline polymer with crystallizable blocks may be a block or multiblock copolymer It may be obtained by polymerizing a monomer comprising reactive (or ethylenic) double bonds or by polycondensation. When the at least one semi-crystalline polymer of the present disclosure is a polymer with crystallizable side chains, these side chains may be in random or statistical form.

In another embodiment, the at least one semi-crystalline polymer is of synthetic origin. In one embodiment, the at least one semi-crystalline polymer according to the present disclosure may lack a polysaccharide skeleton.

By way of non-limiting example, the at least one semi-crystalline polymer that may be used in the compositions according to the present disclosure may be chosen from:
block copolymers of polyolefins of controlled crystallization, such as those whose monomers are described in European Patent Application No. EP 0 951 897,
polycondensates, for example, aliphatic and aromatic polyester polycondensates and aliphatic/aromatic polyester polycondensates,
homopolymers or copolymers bearing at least one crystallizable side chain and homopolymers and copolymers bearing at least one crystallizable block in the skeleton, for instance those described in U.S. Pat. No. 5,156,911,
homopolymers or copolymers bearing at least one crystallizable side chain, for example those bearing at least one fluoro group, such as those described in Patent Publication No. WO 01/19333,
and mixtures thereof.

In the third and fourth examples, the crystallizable side chain(s) or block(s) is (are) hydrophobic.

A) Semi-Crystalline Polymers Comprising Crystallizable Side Chains

By way of non-limiting example, the semi-crystalline polymers comprising crystallisable side chains include those defined in U.S. Pat. No. 5,156,911 and Patent Publication No. WO 01/19333. These polymers are homopolymers or copolymers comprising from 50% to 100% by weight of units resulting from the polymerization of at least one monomer bearing a crystallizable hydrophobic side chain. These homopolymers or copolymers may be of any nature, provided that they meet the conditions mentioned hereinbelow, for example, the characteristic of being soluble or dispersible in the fatty phase when heated above their melting point. They can result:
from the polymerization, for example the free-radical polymerization, of at least one monomer comprising reactive or ethylenic double bond(s) with respect to polymerization, for example a vinyl, (meth)acrylic or allylic group,
from the polycondensation of at least one monomer bearing co-reactive groups (carboxylic acid, sulfonic acid, alcohol, amine, and isocyanate), such as, for example, polyesters, polyurethanes, polyethers, polyureas, and polyamides.

In general, the crystallizable units (chains or blocks) of the at least one semi-crystalline polymer according to the present disclosure may be derived from monomer(s) comprising crystallizable block(s) or chain(s), used for manufacturing the at least one semi-crystalline polymer. These polymers may be chosen, for example, from homopolymers and copolymers resulting from the polymerization of at least one monomer comprising at least one crystallizable chain that may be represented by formula X: 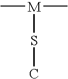
wherein:
M is an atom of the polymer skeleton,
—S is a spacer, and
—C is a crystallizable group

The crystallizable chains “—S—C” may be aliphatic or aromatic, and optionally fluorinated or perfluorinated. “S” may be chosen from linear, branched, and cyclic (CH₂)ₙ, (CH₂CH₂O)ₙ, and (CH₂O) groups, with n being an integer ranging from 0 to 22. In one embodiment, “S” is a linear group. In another embodiment, “S” and “C” are different.

As used herein, the term “alkyl” is understood to mean a saturated group, for example of C₈ to C₂₄, except where otherwise mentioned.

When the crystallizable chains are hydrocarbon-based aliphatic chains, they comprise hydrocarbon-based alkyl chains comprising at least 11 carbon atoms and not more than 40 carbon atoms, for example not more than 24 carbon atoms. They may be aliphatic chains or alkyl chains comprising at least 12 carbon atoms, for example C₁₄-C₂₄ alkyl chains, such as C₁₆-C₂₂ alkyl chains. When they are fluoroalkyl or perfluoroalkyl chains, they may comprise at least 11 carbon atoms, at least 6 carbon atoms of which are fluorinated.

Non-limiting examples of semi-crystalline homopolymers or copolymers comprising crystallizable chain(s) include those resulting from the polymerization of at least one of the following monomers: (meth)acrylates of saturated alkyls, the alkyl group being C₁₄ to C₂₄; perfluoroalkyl(meth)acrylates with a C₁₁ to C₁₅ perfluoroalkyl group; N-alkyl(meth)acrylamides, the alkyl group being C₁₄ to C₂₄ with or without a fluorine atom; vinyl esters comprising alkyl or perfluoroalkyl chains, the alkyl group being C₁₄ to C₂₄ and the perfluoroalkyl chain comprising at least 6 fluorine atoms per chain; vinyl ethers comprising alkyl or perfluoroalkyl chains, the alkyl group being C₁₄ to C₂₄ and the perfluoroalkyl chain comprising at least 6 fluorine atoms per chain; C₁₄ to C₂₄ α-olefins such as, for example, octadecene, para-alkylstyrenes with an alkyl group comprising from 12 to 24 carbon atoms, and mixtures thereof.

When the at least one semi-crystalline polymer results from a polycondensation, the hydrocarbon-based and/or fluorinated crystallizable chains as defined above are borne by a monomer that may be a diacid, a diol, a diamine, or a diisocyanate.

When the at least one semi-crystalline polymer that is used in compositions according to the present disclosure is a copolymer, it may additionally comprise from 0 to 50% of groups Y or Z resulting from the copolymerization:

α) of Y, which is a polar or non-polar monomer or a mixture of the two:

When Y is a polar monomer, it may be chosen from monomers bearing polyoxyalkylenated groups, such as oxyethylenated and/or oxypropylenated groups; hydroxyalkyl(meth)acrylates, for instance hydroxyethyl acrylate; (meth)acrylamide; N-alkyl(meth)acrylamides; N,N-dialkyl(meth)acrylamides, such as, for example, N,N-diisopropylacrylamide and N-vinylpyrrolidone (NVP); N-vinylcaprolactam; monomers bearing at least one carboxylic acid group, for instance (meth)acrylic acid, crotonic acid, itaconic acid, maleic acid, and fumaric acid, or bearing a carboxylic acid anhydride group, for instance maleic anhydride; and mixtures thereof.

When Y is a non-polar monomer, it may be chosen from linear, branched, and cyclic alkyl(meth)acrylate esters, vinyl esters, alkyl vinyl ethers, α-olefins, styrenes and styrenes substituted with a C₁ to C₁₀ alkyl group, for instance α-methylstyrene, and polyorganosiloxane macromonomers containing vinyl unsaturation.

β) of Z, which is a polar monomer or a mixture of polar monomers. In this case, Z has the same definition as the “polar Y” defined above.

In one embodiment, the at least one semi-crystalline polymer comprising a crystallizable side chain is chosen from alkyl(meth)acrylate and alkyl(meth)acrylamide homopolymers with an alkyl group as defined above, such as a C₁₄-C₂₄ alkyl group, copolymers of these monomers with a hydrophilic monomer, such as those different in nature from (meth)acrylic acid, for instance N-vinylpyrrolidone and hydroxyethyl(meth)acrylate, and mixtures thereof.

The at least one semi-crystalline polymer comprising a crystallizable side chain may have a weight-average molar mass Mp ranging from 5,000 to 1,000,000, for example from 10,000 to 800,000, for instance from 15,000 to 500,000, such as from 100,000 to 200,000.

As examples of semi-crystalline polymers that may be used in the compositions according to the present disclosure, non-limiting mention may be made of the Intelimer® products from the company Landec described in the brochure “Intelimer® polymers”, Landec IP22 (Rev. 4-97). These polymers are in solid form at room temperature (25° C.). They bear crystallizable side chains and have the above formula X.

For example, the Intelimer® product IPA 13-1 from the company Landec may be chosen. This product is a polystearyl acrylate with a molecular weight of about 145,000 and a melting point of 49° C.

The at least one semi-crystalline polymer may also be chosen from the polymers described in Examples 3, 4, 5, 7, and 9 of U.S. Pat. No. 5,156,911, comprising a —COOH group, resulting from the copolymerization of acrylic acid and of a C₅ to C₁₆ alkyl (meth)acrylate with a melting point ranging from 20° C. to 35° C., and for example from the copolymerization:
of acrylic acid, of hexadecyl acrylate, and of isodecyl acrylate in a 1/16/3 ratio,
of acrylic acid and of pentadecyl acrylate in a 1/19 ratio,
of acrylic acid, of hexadecyl acrylate, and of ethyl acrylate in a 2.5/76.5/20 ratio,
of acrylic acid, of hexadecyl acrylate, and of methyl acrylate in a 5/85/10 ratio,
of acrylic acid and of polyoctadecyl(meth)acrylate in a 2.5/97.5 ratio.

It is also possible to use the polymer Structure “O” from National Starch, such as the product described in U.S. Pat. No. 5,736,125 with a melting point of 44° C.

The at least one semi-crystalline polymer may be a semi-crystalline polymer with crystallizable pendent chains comprising fluoro groups, as described in Examples 1, 4, 6, 7, and 8 of Patent Publication No. WO 01/19333.

It is also possible to use the semi-crystalline polymers obtained by copolymerization of stearyl acrylate and of acrylic acid or of NVP, as described in U.S. Pat. No. 5,519,063 and European Patent Application No. EP 0 550 745.

It is also possible to use the semi-crystalline polymers obtained by copolymerization of behenyl acrylate and of acrylic acid or of NVP, as described in U.S. Pat. No. 5,519,063 and European Patent Application No. EP 0 550 745.

B) Polymers Bearing at Least One Crystallizable Block in the Skeleton

Polymers that are soluble or dispersible in the fatty phase by heating above their melting point may also be used in compositions according to the present disclosure. These polymers may be block copolymers comprising at least two blocks of different chemical nature, one of which is crystallizable.

The polymer bearing at least one crystallizable block in the skeleton may be chosen from block copolymers of olefin or of cycloolefin comprising a crystallizable chain, for instance those derived from the block polymerization of:
cyclobutene, cyclohexene, cyclooctene, norbornene (i.e., bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, and mixtures thereof,
with ethylene, propylene, 1-butene, 3-methyl-1-butene, 1-hexene, 4-methyl-1-pentene, 1-octene, 1-decene or 1-eicosene, and mixtures thereof,
and, for example, copoly(ethylene/norbornene) blocks and (ethylene/propylene/ethylidene-norbornene) block terpolymers. Those resulting from the block copolymerization of at least two C₂-C₁₆, for instance C₂-C₁₂, α-olefins such as those mentioned above and, for example, block bipolymers of ethylene and of 1-octene may also be used.

The polymer bearing at least one crystallizable block in the skeleton may be chosen from copolymers comprising at least one crystallizable block, the rest of the copolymer being amorphous at room temperature. These copolymers may also comprise two crystallizable blocks of different chemical nature.

Non-limiting examples of copolymers that may be mentioned include those that simultaneously comprise at room temperature a crystallizable block and an amorphous block that are both hydrophobic and lipophilic, sequentially distributed. Non-limiting mention may be made, for example, of polymers comprising one of the crystallizable blocks and one of the amorphous blocks below:
blocks that are crystallizable by nature, of polyester type, for instance poly(alkylene terephthalate), and of polyolefin type, for instance polyethylenes or polypropylenes; and
amorphous and lipophilic blocks, for instance amorphous polyolefins and copoly(olefin)s such as poly(isobutylene), hydrogenated polybutadiene, and hydrogenated poly(isoprene).

As examples of such copolymers comprising a crystallizable block and an amorphous block, non-limiting mention may be made of:
α) poly(ε-caprolactone)-b-poly(butadiene) block copolymers, used, for example, hydrogenated, such as those described in the article “Melting behavior of poly(-caprolactone)-block-polybutadiene copolymers” by S. Nojima, Macromolecules, 32, 3727-3734 (1999),
β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34,117-123 (1995),
γ) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),
δ) the poly(ethylene)-b-poly(ethylethylene) block copolymers cited in the general article “Crystallization in block copolymers” by I. W. Hamley, Advances in Polymer Science, Vol. 148, 113-137 (1999).
   C) Aliphatic, Aromatic, and Aliphatic/Aromatic Polyester Polycondensates

The polyester polycondensates may be chosen from aliphatic polyesters. Their molar mass may be greater than or equal to 200 and less than or equal to 10,000, for example greater than or equal to 300 and less than or equal to 5,000, such as greater than or equal to 500 and less than or equal to 2,000 g/mol.

The polyester polycondensates may be chosen from polycaprolactones. For example, the polycaprolactones may be chosen from ε-caprolactone homopolymers. The homopolymerization may be initiated with a diol, for example a diol comprising from 2 to 10 atoms, such as diethylene glycol, 1,4-butanediol, and neopentyl glycol.

For example, polycaprolactones may be used, such as those sold under the name CAPA® 240 (melting point of 68° C. and molecular weight of 4,000), 223 (melting point of 48° C. and molecular weight of 2,000), 222 (melting point of 48° C. and molecular weight of 2,000), 217 (melting point of 44° C. and molecular weight of 1,250), 2125 (melting point of 45° C. and molecular weight of 1,250), 212 (melting point of 45° C. and molecular weight of 1,000), 210 (melting point of 38° C. and molecular weight of 1,000), 205 (melting point of 39° C. and molecular weight of 830) by the company Solvay, and PCL-300 and PCL-700 by the company Union Carbide.

In certain embodiments, the CAPA® 2125 whose melting point ranges from 35° C. to 45° C. and whose molecular weight is equal to 1,250 may be used.

The at least one semi-crystalline polymers may or may not be partially crosslinked, provided that the degree of crosslinking does not interfere with their dissolution or dispersion in the fatty phase by heating above their melting point. It may be a chemical crosslinking, by reaction with a multifunctional monomer during the polymerization. It may also be a physical crosslinking that may, in this case, be due either to the establishment of hydrogen or dipolar bonds between groups borne by the polymer, such as, for example, the dipolar interactions between carboxylate ionomers, these interactions being of small amount and borne by the polymer skeleton; or to a phase separation between the crystallizable blocks and the amorphous blocks borne by the polymer.

In certain embodiments, the at least one semi-crystalline polymer in the compositions according to the present disclosure are non-crosslinked.

In practice, the at least one semi-crystalline polymer may be present in an amount ranging from 0.1%to 80% by weight, for example from 0.5% to 40% by weight, such as from 3% to 30% by weight, relative to the total weight of the composition. In certain embodiments, the at least one semi-crystalline polymer may be present from 5% to 25% by weight, relative to the total weight of the composition.

Particulate Phase

The compositions according to the present disclosure may comprise at least one pigment and/or at least one filler.

The at least one pigment may be white or colored, mineral and/or organic, and of interference or non-interference type. Among the mineral pigments that may be used, non-limiting mention may be made of titanium dioxide, optionally surface-treated, zirconium oxide, cerium oxide, zinc oxide, iron oxide (black, yellow, and red), chromium oxide, manganese violet, ultramarine blue, chromium hydrate, and ferric blue. Among the organic pigments that may be used, non-limiting mention may be made of carbon black, pigments of the type such as organic lakes of barium, strontium, calcium, and aluminum, including those submitted for certification by the Food and Drug Administration (FDA) (e.g., D&C or FD&C) and those exempt from FDA certification, for instance lakes based on cochineal carmine. The at least one pigment can be present in an amount ranging from 0.1% to 50%, for example from 0.5% to 35%, such as from 2% to 25% by weight of active material, relative to the total weight of the composition.

The nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the type mentioned above, as well as nacreous pigments based on bismuth oxychloride. They can be present in an amount ranging from 0% to 25% by weight of active material, for example from 0.1% to 15% by weight of active material, relative to the total weight of the composition (if present). Pigments with goniochromatic properties and/or pigments with a metallic effect, as described, for example, in the French Patent Application filed under the number FR 0 209 246, the content of which is incorporated by reference herein, may thus be used.

The fillers may be mineral or organic, and lamellar or spherical. Non-limiting mention may be made of talc, mica, silica, kaolin, Nylon® powder (Orgasol® from Atochem), poly-β-alanine powder, polyethylene powder, powders of tetrafluoroethylene polymers (Teflon®), lauroyllysine, starch, boron nitride, hollow microspheres such as Expancel® (Nobel Industrie), Polytrap® (Dow Corning), silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass microcapsules, ceramic microcapsules, metal soaps derived from carboxylic organic acids comprising from 8 to 22 carbon atoms, such as from 12 to 18 carbon atoms, for example zinc stearate, magnesium stearate, lithium stearate, zinc laurate, and magnesium myristate.

The compositions according to the present disclosure may comprise particles that are solid at room temperature, dispersed in the physiologically acceptable medium, introduced into the compositions in the form of a colloidal dispersion, as described in Patent Publication No. WO 02/39961, the content of which is incorporated by reference herein.

The compositions according to the present disclosure may comprise at least one dispersant. The at least one dispersant may serve to protect the dispersed filler or pigment particles against their agglomeration or flocculation. The concentration of dispersant that may be used to stabilize a colloidal dispersion may range from 0.3 to 5 mg/m², for example from 0.5 to 4 mg/m², of surface area of pigment and/or filler particles. This dispersant may be chosen from surfactants, oligomers, polymers, and mixtures thereof, comprising at least one functionality having strong affinity for the surface of the particles to be dispersed. For example, they can physically or chemically attach to the surface of the pigments. These dispersants may also have at least one functional group that is compatible with or soluble in the continuous medium. For example, esters of 12-hydroxystearic acid, of a C₈ to C₂₀ fatty acid, and of a polyol, for instance glycerol and diglycerol, may be used, such as poly(12-hydroxystearic acid) stearate with a molecular weight of about 750 g/mol, for example the product sold under the name Solsperse 21 000 by the company Avecia, polyglyceryl-2 dipolyhydroxystearate (CTFA name) sold under the reference Dehymyls PGPH by the company Henkel, and polyhydroxystearic acid, for example the product sold under the reference Arlacel P100 by the company Uniqema, and mixtures thereof.

As other dispersants that may be used in the compositions according to the present disclosure, non-limiting mention may be made of quaternary ammonium derivatives of polycondensed fatty acids, for instance Solsperse 17 000 sold by the company Avecia, and mixtures of polydimethylsiloxane/oxypropylene, such as those sold by the company Dow Corning under the references DC2-5185 and DC2-5225 C.

The polydihydroxystearic acid and the 12-hydroxystearic acid esters may be used, for example, in hydrocarbon-based and fluorinated media, whereas the mixtures of oxyethylene/oxypropylene dimethylsiloxane may be used, for example, in silicone media.

Additives and Galenical Forms

The compositions according to the present disclosure may also comprise at least one cosmetic, dermatological, hygiene, and/or dermatological active agent such as those conventionally used.

Among the cosmetic, dermatological, hygiene, and/or pharmaceutical active agents that may be used in the compositions according to the present disclosure, non-limiting mention may be made of moisturizers, vitamins, essential fatty acids, sphingolipids, and sunscreens. These active agents may be used in a usual amount for a person skilled in the art, for example in an amount ranging from 0% to 20%, such as from 0.001% to 15% by weight, relative to the total weight of the composition.

The compositions according to the present disclosure may also comprise any other additive usually used in such compositions, such as water, gelling agents, water-soluble dyes, antioxidants, fragrances, preserving agents, and essential oils.

Needless to say, a person skilled in the art will take care to select this or these optional additional compound(s), and/or the amount thereof, such that the advantageous properties of the compositions according to the present disclosure are not, or are not substantially, adversely affected by the envisaged addition.

In certain embodiments, the compositions according to the present disclosure may be prepared in the usual manner by a person skilled in the art. They may be in the form of a cast product, for example in the form of a stick or tube, or in the form of a dish that may be used by direct contact or with a sponge. For example, they may be in the form of a cast foundation, a cast makeup rouge, a cast eyeshadow, a lipstick, base or balm to care for the lips, or a concealer product. They may also be in the form of a soft paste or alternatively a more or less fluid gel or cream, or a liquid, packaged in a tube. They may then constitute foundations, lipsticks, antisun products, and/or skin-coloring products.

The compositions according to the present disclosure may be anhydrous and, in this case, contain less than 5% of water relative to the total weight of the composition.

These compositions for topical application may constitute cosmetic, dermatological, hygiene, and/or pharmaceutical compositions for protecting, treating, and/or caring for the face, the neck, the hands, and/or the body (for example care creams, antisun oils, and body gels), makeup compositions (for example makeup gels, creams, and sticks), and/or compositions for artificially tanning and/or for protecting the skin.

The compositions according to the present disclosure may be in the form of dermatological and/or care compositions for the skin and/or the integuments or in the form of antisun compositions or body hygiene compositions, for example in deodorant form. It may then be in uncolored form. It may then be used as a care base for the skin, the integuments and/or the lips (lip balms, for protecting the lips against the cold and/or sunlight, and/or the wind, or a care cream for the skin, the nails, and/or the hair).

The compositions according to the present disclosure may be obtained by heating the various constituents to the melting point of the highest-melting waxes, followed by casting of the molten mixture in a mould (dish or finger stall). They may also be obtained by extrusion, as described in European Patent Application No. EP 0 667 146.

The disclosure may be understood more clearly with the aid of the non-limiting examples that follow, which constitute preferred embodiments of the compositions according to the disclosure. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE 1

### Lipstick

| Chemical name | Mass percentage |
|---|---|
| Dispersion of acrylate polymer in hydrogenated | 30 |
| polyisobutene, surface-stabilized with Kraton G1701 | |
| 2-Decyltetradecanoic acid triglyceride | 2.02 |
| Dilinoleyl diol dimer/dilinoleic dimer copolymer | 10 |
| (Lusplan DD-DA 5 from NFC) | |
| Octyldodecanol | 9 |
| BHT | 0.07 |
| Mixture of parabens | 0.4 |
| Polycaprolactone of MW 1,250 g/mol | 9 |
| Vinylpyrrolidone/eicosene copolymer | 6 |
| Microcrystalline wax | 10 |
| Polyethylene wax (MW 500) | 2 |
| Polymethylene wax of m.p. 40° C. | 10 |
| Stearyl alcohol | / |
| Pigments | 6.03 |
| Dimethicone-coated silica | 5 |
| Fragrance | 0.48 |
| TOTAL | 100 |

Synthesis of the Dispersion of Polymer Particles:

A dispersion of non-crosslinked copolymer of methyl acrylate and of acrylic acid in an 85/15 ratio, in heptane, was prepared according to the method of Example 1 of European Patent Application No. 0 749 746. When the polymerization was complete, hydrogenated polyisobutene was added and the heptane was distilled off under vacuum.

A dispersion of poly(methyl acrylate/acrylic acid) particles surface-stabilized in the hydrogenated polyisobutene with a polystyrene/copoly(ethylene-propylene) diblock copolymer sold under the name Kraton G1701, having a solids content of 21% by weight and a mean particle size equal to 150 nm, was thus obtained.

Procedure for Preparing the Lipstick

All the starting materials were weighed out in an oil-circulated jacketed heating pan and were then heated with stirring (turbomixer).

After total melting of the materials and homogenization of the mixture, it was ground 5 times in succession on a three-roll mill. The paste obtained was left to stabilize for 24 hours at 20° C. and then packaged in heating bags.

Measurement of the Resistance Index

The resistance index of Example 1, measured according to the protocol described previously, was equal to 54.

What is claimed is:

1. A composition comprising particles of at least one polymer dispersed in a fatty phase, wherein the fatty phase is free of volatile oil or comprises less than 50% by weight of volatile oil, relative to the weight of the fatty phase, and wherein the at least one polymer is such that when it is dispersed in sufficient amount in the composition, the composition is capable of forming a deposit having a resistance index of greater than or equal to 30%.

2. The composition according to claim 1, wherein the composition is capable of forming a deposit having a resistance index of greater than or equal to 40%.

3. The composition according to claim 2, wherein the composition is capable of forming a deposit having a resistance index of greater than or equal to 50%.

4. The composition according to claim 1, wherein the mean gloss of the composition measured at 20° C. is greater than or equal to 30 out of 100.

5. The composition according to claim 4, wherein the mean gloss of the composition measured at 20° C. is greater than or equal to 60 out of 100.

6. The composition according to claim 1, wherein the particles are solid and insoluble in the fatty phase at a temperature of 25° C.

7. The composition according to claim 1, wherein the at least one polymer is not a wax.

8. The composition according to claim 1, wherein the particles have a mean size ranging from 5 nm to 800 nm.

9. The composition according to claim 1, wherein the at least one polymer is film-forming.

10. The composition according to claim 9, wherein the at least one polymer is a hydrocarbon-based polymer.

11. The composition according to claim 1, wherein the particles are insoluble in water-soluble alcohols.

12. The composition according to claim 1, wherein the at least one polymer is chosen from polyurethanes, polyurethane-acrylics, polyureas, polyureas/polyurethanes, polyester-polyurethanes, polyether-polyurethanes, polyesters, polyester amides, fatty-chain polyesters, alkyds, acrylic polymers, acrylic copolymers, vinyl polymers, vinyl copolymers, acrylic-silicone copolymers, polyacrylamides, silicone polymers, fluoro polymers, and mixtures thereof.

13. The composition according to claim 1, wherein the at least one polymer is chosen from meth(acrylic)/(meth)acrylate copolymers.

14. The composition according to claim 13, wherein the at least one polymer is chosen from acrylic/acrylate copolymers such that the mass ratio of the acrylic units and of the acrylate units ranges from 0.1% to 40%.

15. The composition according to claim 14, wherein the at least one polymer is chosen from acrylic/acrylate copolymers such that the mass ratio of the acrylic units and of the acrylate units ranges from 5% to 20%.

16. The composition according to claim 1, wherein the at least one polymer is present, as a solid, in an amount ranging from 5% to 40% by weight, relative to the total weight of the composition.

17. The composition according to claim 16, wherein the at least one polymer is present, as a solid, in an amount ranging from 8% to 30% by weight, relative to the total weight of the composition.

18. The composition according to claim 1, further comprising at least one stabilizer chosen from block polymers, grafted polymers, random polymers, and mixtures thereof.

19. The composition according to claim 18, wherein the at least one stabilizer is chosen from grafted-block and block polymers comprising at least one block resulting from the polymerization of diene and at least one block of a vinyl polymer.

20. The composition according to claim 19, wherein the at least one stabilizer is chosen from diblock polymers.

21. The composition according to claim 1, further comprising at least one apolar or sparingly polar oil.

22. The composition according to claim 21, wherein the at least one apolar or sparingly polar oil is present in an amount ranging from 5% to 80% by weight, relative to the total weight of the composition.

23. The composition according to claim 22, wherein the at least one apolar or sparingly polar oil is present in an amount ranging from 10% to 60% by weight, relative to the total weight of the composition.

24. The composition according to claim 23, wherein the at least one apolar or sparingly polar oil is present in an amount ranging from 15% to 30% by weight, relative to the total weight of the composition.

25. The composition according to claim 21, wherein the at least one apolar or sparingly polar oil is chosen from hydrocarbon-based apolar oils.

26. The composition according to claim 25, wherein the hydrocarbon-based apolar oils are chosen from oils with a molar mass ranging from 300 to 900 g/mol.

27. The composition according to claim 26, wherein the hydrocarbon-based apolar oils are chosen from oils with a molar mass ranging from 350 to 800 g/mol.

28. The composition according to claim 25, wherein the hydrocarbon-based apolar oils are chosen from linear and branched hydrocarbons.

29. The composition according to claim 28, wherein the linear and branched hydrocarbons are chosen from liquid paraffin, liquid petroleum jelly, liquid naphthalene, hydrogenated polyisobutene, isoeicosane, squalane, decene/butene copolymers, and mixtures thereof.

30. The composition according to claim 1, further comprising at least one oil with a molar mass ranging from 650 to 10,000 g/mol and present in an amount ranging from 2% to 30% by weight, relative to the total weight of the composition.

31. The composition according to claim 30, wherein the at least one oil with a molar mass ranging from 650 to 10,000 g/mol is present in an amount ranging from 5% to 15% by weight, relative to the total weight of the composition.

32. The composition according to claim 1, further comprising at least one oil with a molar mass ranging from 750 to 7,500 g/mol and present in an amount ranging from 2% to 30% by weight, relative to the total weight of the composition.

33. The composition according to claim 32, wherein the at least one oil with a molar mass ranging from 750 to 7,500 g/mol is present in an amount ranging from 5% to 15% by weight, relative to the total weight of the composition.

34. The composition according to claim 1, further comprising at least one pulverulent dyestuff chosen from pigments, nacres, flakes, and mixtures thereof.

35. The composition according to claim 1, further comprising at least one dispersant chosen from poly(12-hydroxystearic acid stearate), poly(12-hydroxystearic acid), diglyceryl 2-dipolyhydroxystearate, and mixtures thereof.

36. The composition according to claim 1, wherein the composition is in anhydrous form.

37. The composition according to claim 1, wherein the composition is in a form chosen from a product for caring for and/or making up the skin and/or the lips.

38. The composition according to claim 1, wherein the composition is in a form chosen from a foundation, a makeup rouge, an eyeshadow, a lipstick, a care base for the lips, a care balm for the lips, a concealer product, an eyeliner, and a mascara.

39. A method of obtaining a glossy, non-migrating makeup with good staying power comprising
applying a composition comprising particles of at least one polymer dispersed in a fatty phase, wherein the fatty phase is free of volatile oil or comprises less than 50% by weight of volatile oil, relative to the weight of the fatty phase, and wherein the at least one polymer is such that when it is dispersed in sufficient amount in the composition, the composition is capable of forming a deposit having a resistance index of greater than or equal to 30%.
